(19) United States
(12) Patent Application Publication
(10) Pub. No.: US 2016/0113549 A1
(43) Pub. Date: Apr. 28, 2016

(54) DEVICE FOR MEASURING THE CIRCUMFERENCE OF AN OBJECT, IN PARTICULAR A LIMB

(71) Applicant: JUST A NEW HEALTH, Forest (BE)

(72) Inventor: Joseph Harfouche, Forest (BE)

(73) Assignee: JUST A NEW HEALTH, Forest (BE)

(21) Appl. No.: 14/894,314

(22) PCT Filed: May 28, 2014
(86) PCT No.: PCT/EP2014/061165

§ 371 Date: Nov. 25, 2015

(30) Foreign Application Priority Data
May 30, 2013 (BE) 2013/0380

(51) Int. Cl. A61B 5/107; A61B 5/00

(52) U.S. Cl. CPC A61B 5/1075; A61B 5/418

(57) ABSTRACT

The application relates to a device for the iterative measurement of the circumference of an object, in particular a limb, comprising: a first longitudinal graduated measuring element having a first longitudinal direction, arranged to be placed alongside said object, in particular alongside said limb and defining a slide, a distal strap and a proximal strap each located in a plane substantially perpendicular to said longitudinal direction, and a slider engaging with said slide, linked to a second longitudinal graduated measuring element, said slider having a hole for the slide to pass through and being positioned between said distal strap and said proximal strap.

The present invention relates to a device for the iterative measurement of the circumference of an object, in particular a limb, comprising:
a first longitudinal graduated measuring element having a first longitudinal direction, arranged to be affixed alongside said object, in particular alongside said limb and defining a slide,
a distal strap and a proximal strap fitted each in a plane substantially perpendicular to said longitudinal direction, and
a slider cooperating with said slide, connected to a second longitudinal graduated measuring element, said slider having a slide passage orifice and being positioned between the distal strap and the proximal strap.

A measurement of the circumference of limbs, such as an arm or a leg, is in particular recommended for the profile and monitoring of physical therapy treatment, for example to treat lymphedemas, i.e., swelling of part of the body following a build-up of lymphatic fluid in the interstitial tissues. Such swelling appears when the lymphangions that make up the lymphatic vessels are damaged or nonfunctional (primary lymphedema) or when the lymphatic vessels and cells are damaged or obstructed and when lymph nodes have been removed (secondary lymphedema).

More particularly, secondary lymphedemas are a consequence of damage or trauma for example caused by an accident, surgery, serious infection, radiation therapy or other causes.

This swelling essentially relates to the upper and lower limbs, for example the arms, feet, legs, thighs and hands, but can also occur in other parts of the body such as the neck, abdomen, back or breasts. It should be noted that secondary lymphedema of the upper limbs is primarily caused by surgical treatment of the axilla in breast cancer, consisting of an ablation of the ganglions of the armpit.

In order to determine the extent to which a lymphedema (primary or secondary) must be treated, its evolution should be monitored. As an example, if we consider secondary lymphedema of the upper limbs, the clinical practice guidelines for the care and treatment of breast cancer (Clinical practice guidelines for the care and treatment of breast cancer, Canadian Medical Association Journal) recommends measuring the branchial circumference at four points: the metacarpal-phalangeal articulations, the wrists, 10 cm below and 15 cm above the lateral epicondyle points (elbow). It is considered that a difference of more than 2 cm in the circumference between two measurements of one of these four measuring points justifies treatment for lymphedema. A difference of more than 2 cm in the circumference between a limb (for example the right arm) having a lymphedema and corresponding limb (for example the left arm) not having a lymphedema also indicates that treatment should be done for the swelling.

It is therefore necessary to have a device or a medical instrument that makes it possible to perform periodic measurements of the circumference of limbs in the same location to be able to decide whether lymphedema treatment is applicable. In particular, one should have a measuring tool that allows precise and reliable measurement, since the margin of error must be small and only approximately several millimeters, preferably approximately less than 5 mm, and still more preferably approximately less than 2 mm.

Measuring limb circumference is also indicated to see a decrease in the volume of the skeletal muscles and monitor the evolution thereof. Such a decrease in the muscle volume (or loss of muscle mass) can for example be due to amyotrophy (atrophy and/or disappearance of striated muscle fiber), sarcopenia (geriatric syndrome) or myopathies (neuromuscular disease). These pathologies require monitoring, and in particular treatment with physical therapy, during which a precise measurement of the circumference of the limb comprising the considered muscle is essential. Here as well, the evolution of the limb circumference can be monitored by comparing two measurements done in a same location after a predetermined length of time or by comparing the circumferences of a “healthy” limb and a corresponding limb having a decrease in muscle volume (loss of muscle mass).

Measuring limb circumference is also indicated to see a decrease in volume when following a diet and during anti-cellulite treatments, during which refinement of the limb is expected.

Measuring limb circumference is also indicated to see a change in volume during training as part of a strength training program, where an increase in muscle volume is expected or hoped for.

It is of course understood that any other condition or pathology causing a variation in limb circumference falls within the scope of the present invention.

Furthermore, the Directives of the National Institute for Health and Disability Insurance (INAMI) recommend that practitioners (physicians, physical therapists, etc.) perform measurements every 4 cm along the limb having swelling or suffering from a decrease in muscle volume. A significant number of measurements must therefore be done, and it is thus necessary to have a measuring tool which, once positioned along the limb, makes it possible to read or perform measurements at regular intervals along the limb reliably, quickly, precisely and reproducibly.

A lymphedema measuring device is for example known from document GB 2,452,256, which discloses a measuring device comprising a telescopic ruler (extensible) formed by a series of segments, two successive segments having a smaller section from a proximal end to a distal end of said graduated ruler. Furthermore, this device comprises a distal strap and a proximal strap designed both to be fixed to the device at the ends of a limb, or at least in two separate zones of the limbs, such that the telescopic ruler is placed along the limb where the measurements must be taken. According to the device described in this prior document, the circumference measurements can be done using a graduated sliding strip that can be moved alongside segments of the graduated telescoping ruler using the slider. Winding this sliding strip around a portion of the limb makes it possible to define the circumference of said considered portion in a precise location on the limb, this location being defined owing to the graduation of the graduated telescopic ruler.

Unfortunately, such a device for measuring the circumference of limbs comprising a telescoping ruler means that the segments making up said ruler must be rigid enough to be able to fit into one another or on one another. Such rigidity, which is mandatory according to the device of document GB 2,452,256, makes it difficult to place such a measuring device alongside all of the limbs and according to all pathologies while ensuring close contact, i.e., against the skin, on the surface and all along the limbs such that the measurements are precise regardless of the situation encountered. Indeed, the rigidity of the graduated ruler makes it difficult to place and makes its application more difficult against and alongside a limb following that same limb along any curves it may have (elbow, knee, etc.) and/or any protuberances and/or swelling, which are for example due to the very morphology of the limb in question, or the presence of edema.

Furthermore, since the different segments of the ruler have variable sections, i.e., successive sections whose length from one segment to the next decreases from a proximal end to a distal end of said ruler, the slider making it possible to move the graduated sliding strip must necessarily have an opening or passage orifice of the slider able to engage over the widest of the segments. As a result, when the slider is positioned at a segment having a smaller width than that of the widest of the segments, a certain “play” or “float” between said slider and said segment of said graduated ruler is observed, which makes any measurement approximate and imprecise. In fact, such “play” or “float” can cause measurement imprecisions of approximately one to several centimeters, the slider tending to have an inclined position relative to the axis defined by said graduated ruler, which inevitably implies imprecision of the measurement done. However, when the decision whether to treat a pathology such as a lymphedema is based on a small variation in the circumference of the limb of approximately 2 cm, such an imprecision of approximately a centimeter is completely unacceptable.

Furthermore, a telescopic ruler necessarily has a limitation in terms of length. Indeed, in order for the measuring device to keep a reasonable size, the number of telescopic segments must not be excessive, which would lead to too many nestings or too many superpositions, which, ultimately, would lead to a bulky device that is difficult to handle. Consequently, although the device must be able to be affixed along a relatively long limb, for example along a lower limb such as a leg, this would mean that the first segment of the ruler must be wide enough to be able to receive the other telescopic segments having a smaller size or width. Such a width of the first segment would be problematic inasmuch as it could prevent correct positioning of the measuring device alongside a limb.

The invention aims to offset the drawbacks of the state of the art by procuring a device for measuring limb circumference that makes it possible to perform quick, precise, reliable and reproducible measurements irrespective of the limb in question, whether the latter is short or long, has curves and/or protuberances, and at any location along the limb in question. Furthermore, the invention also aims to procure a measuring device that has a reasonable size, that is easy to handle, light and not very bulky, so as for example to be easily stored in the pocket of a piece of clothing, which is also a definite advantage in the medical field, where healthcare professionals, moving from room to room or from one office to another to see their patients, frequently store their equipment in the pockets of their work shirt.

To resolve these problems of the state of the art, the invention provides an iterative measuring device for the circumference of an object, in particular a limb, as indicated in the beginning of this document, characterized in that said slide is a flexible slide having a constant section over its entire length and in that said passage orifice is delimited by four walls, at least two of which are parallel to one another and which each form a guide wall for the slider along the slide so as to fit said second longitudinal graduated measuring element in a plane perpendicular to said first longitudinal direction.

Such a device as recommended according to the invention is particularly advantageous and makes it possible to measure the circumference of any limb in any location on that limb, quickly, precisely, reproducibly and reliably, irrespective of whether the limb in question has curves and/or protuberances.

Indeed, according to the present invention, a precise measurement of the circumference of the limb can be obtained under any circumstances, since said at least one slider is arranged such that it closely and perpendicularly follows the first graduated measuring element, which defines a slide having a constant section. This is made possible by the fact that the passage orifice of the slider is limited by four walls, at least two of which are parallel to one another and which each form a guide wall of the slider along the slide so as to fit said second longitudinal graduated measuring element into a plane perpendicular to said first longitudinal direction. The slide can then act as a veritable guide (pilot) of the slider, which itself then guides the second measuring element, consequently also being vertically perpendicular relative to the first graduated measuring element (i.e., relative to the slide), along which it can move longitudinally while maintaining this perpendicularity continuously.

Furthermore, since the first graduated measuring element (or slide) is made from a flexible material, it can be affixed along the limbs perfectly over its entire length, directly against the skin, even if these limbs have protuberances and/or hollows. Furthermore, since the first graduated measuring element is formed and manufactured from a flexible material, it can easily be wound and/or folded, which makes it possible to minimize the size thereof and makes it easier to store, transport and handle.

Within the meaning of the present invention, the terms “flexible”, “flexible slide” or “slide made from a flexible material” refer to a slide whose flexibility allows it to follow, closely and by contact, the surface of an object or a limb whose circumference must be measured, this flexibility also allowing folding, curvature or winding of the slide. However, within the meaning of the present invention, this flexibility must not be excessive so that the width of the slide is not flexible, thereby preventing the slide from remaining substantially planar. For example, the “flexibility” within the meaning of the present invention cannot be tied to a certain elasticity that would tend to allow a deformation of the slide during its placement along a limb.

According to the present invention, precise, reliable and reproducible limb circumference measurements can therefore be done under all circumstances, irrespective of the limb in question or the location of the limb in question, since the measuring device according to the invention makes it possible, by the flexibility of the first graduated measuring element, to perfectly follow the surface of the limb, but also to ensure the perpendicularity of the second graduated measuring element relative to the first graduated measuring element due to the properties of the slider, whose passage orifice for the slide is defined by four walls, at least two of which are parallel to one another and which each form a guide wall for the slider along the slide so as to fit said second longitudinal graduated measuring element into a plane perpendicular to said first longitudinal direction. Such simultaneously precise, reliable and reproducible limb circumference measurements are essential to eliminate any margin of error that could distort diagnoses and call into question the very principle of the measurement of the circumference of limbs as a decision-making tool before beginning—or not beginning—a treatment.

Preferably, in the measuring device according to the invention, said slide and/or said second graduated measuring element can be wound. For example, said slide and/or said second graduated measuring element can assume the form of a tape measure like those used in the sewing field and which are generally made from a flexible plastic material or from paper having with a high tensile strength, which makes it possible to wind these measuring tapes either manually or automatically using an automatic winding system, for example comprising a return spring. Winding the slide and/or said second graduated measuring element has the advantage of minimizing the size of the measuring device when it is not in use, which makes it possible to store it in a relatively compact form and transport it easily. Furthermore, the fact that the slide and/or the second graduated measuring element can be wound allows the operator to unwind only the portion he actually needs when taking a measurement, the unused portion of the slide and/or said second graduated measuring element remaining wound, being relatively compact and not hindering the movements of the operator.

Preferably, in the measuring device according to the invention, said slide and/or said second graduated measuring element is foldable, for example in an S shape. The same advantages as those mentioned above are applicable when such folding of the slide is done.

Advantageously, the measuring device according to the invention comprises a plurality of sliders. According to the invention, an unlimited number of sliders can be added on the slide. When several sliders are available on the slide, one or more operators can perform several measurements simultaneously, which makes it possible to save time, in particular when periodic measurements need to be done at an interval of several centimeters on a bodily segment having a relatively long length, for example a leg. Optionally, additional straps can also be connected to the sliders if the positioning of the slide requires more than two fastening points along the limb.

Preferably, in the measuring device according to the invention, said proximal strap is connected to the slide or to a slider other than that to which said second graduated measuring element is connected, said proximal strap being detachable or not. When the proximal strap is connected to the slide, its position is stationary whereas, advantageously, when it is connected to a slider other than that to which said second graduated measuring element is connected, its position is variable, which makes it possible to choose the location or zone precisely where it will be fastened on the limb in question. Being able to detach the proximal strap from the slide or slider makes it possible to minimize the size of the measuring device when it is not in use, and to charge the proximal strap if, for example, it must have a smaller or larger length or width, which depends on the limb in question.

Advantageously, in the measuring device according to the invention, said passage orifice of said slider has a section similar to the section of the slide. The sections of the slide and the passage orifice of the slider are substantially similar, a perpendicularity of the second measuring element in a plane perpendicular to said first longitudinal direction is even better respected, since similar sections mean that the four walls defining the passage orifice of the slider are in close contact with the slide.

Preferably, in the measuring device according to the invention, said slide is a slide made from a flexible material, for example flexible plastic or paper with a sufficient tensile strength.

Preferably, according to the invention, said first and/or second measuring elements as well as the distal straps and/or proximal straps are made from a material of the plasticized canvas type.

Advantageously, in the measuring device according to the invention, said second measuring element connected to the slider is detachable. For example, the second measuring element can comprise fastening means in the form of a male member of a fastening system on which a female member is fixed situated on the slider. It may also be a fastening means of the Velcro® type or any other type of appropriate fastening means. Being able to detach the second measuring element from the slider makes it possible to minimize the size of the measuring device when it is not in use, as well as to change measuring elements if, for example, said measuring element must have a smaller or larger length or width, which depends on the limb in question.

Preferably, in the measuring device according to the invention, said distal strap is connected to the slide or to a slider other than that to which said second graduated measuring element is connected, said distal strap being detachable or not. When the distal strap is connected to the slide, its position is fixed whereas, advantageously, when is connected to a slider other than that to which said second graduated measuring element is connected, its position is variable, which makes it possible to choose the location or zone precisely where it will be fixed on the limb in question. Being able to detach the distal strap from the slide or slider makes it possible to minimize the size of the measuring device when it is not in use, but also to the change distal strap if, for example, it must have a smaller or larger length or width, which depends on the limb in question.

For example, according to the invention, said proximal strap and/or said distal strap is a collar of the reusable Colson® type, i.e., which can be closed, then opened. It may also be a collar of the single-use Colson® type, i.e., only provided to be closed. In this case, it should be cut in order to detach the measuring device placed around the object whose circumference has been measured.

Advantageously, in the measuring device according to the invention, said distal strap and/or said proximal strap is a graduated or not graduated longitudinal strip having first and second ends each provided with a closing means and having an open position and a closed position, said open position being a position where said first and second ends are spaced apart from one another, while the closed position is a position in which said first end of said longitudinal strip is folded down on said second end.

Preferably, in the measuring device according to the invention, said closing means is chosen from the group made up of a press button, a pushbutton or self-gripping means, for example of the Velcro® type. Such a closing means is particularly practical, since it can be open and closed quickly by a simple pulling or pushing movement. It is of course understood that any other type of appropriate closing means is also covered by the present invention. Advantageously, according to the invention, the end of the slide to which said distal strap is connected further comprises an additional graduated or not graduated longitudinal portion extending in the extension of said slide beyond the zero graduation of the latter. The presence of such an additional portion allows a placement of the distal strap, by using or not using a slider, upstream from the zero graduation such that said distal strap does not prevent precise viewing of the zero graduation. Furthermore, this additional portion can allow, upstream from the zero graduation, the potential placement of additional straps or any other fastening elements and/or additional measuring elements. This additional portion can itself be extended by a removable or non-removable end-piece constituting a stop preventing the sliders from “leaving” said slide extended by this additional portion.

Advantageously, according to the invention, the measuring device further comprises a system for blocking the distal strap and/or the proximal strap. The presence of such blocking systems (or brake system) makes it possible to improve the precision of the measurements done at regular intervals while ensuring maintenance in place of the first measuring element, along which a series of sliders can move that are each connected to a second longitudinal graduated measuring element. It is of course understood that any type of blocking system making it possible to perform such blocking of the distal and/or proximal strap is part of the present invention.

Preferably, according to the invention, when said second graduated measuring element is associated with a winder-type device in order to be stored therein, the weight of the latter is approximately 10 to 50 g, preferably approximately 25 to 30 g, preferably approximately 20 g. It has in fact been determined that such a weight of the winder-type device is appropriate inasmuch as, under the force of gravity, it exerts constant traction making it possible to ensure precision of the measurements done. Indeed, the traction force thus exerted on said second graduated measuring element ensures that the latter is not deformed, but also that it is positioned correctly relative to the first measuring element. The risks of error are consequently minimized during the measurement of the circumference of an object.

Advantageously, according to the invention, when sliders are present along said first measuring element and when it is provided that said first measuring element is wound in a winder to be stored therein, both said first measuring element and said sliders are arranged to be stored simultaneously in the same winder.

Preferably, according to the invention, said second measuring element is provided, at its end not connected to the first measuring element, with a removable or non-removable end-piece comprising a first pair of protrusions extending upward in a direction perpendicular to the plane formed by said second graduated measuring element and/or a second pair of protrusions extending outward substantially in the same plane as that formed by said second graduated measuring element. This is particularly advantageous inasmuch as, on the one hand, said first pair of protrusions forms a guide for the part of said second measuring element engaged therein during measurement of the circumference of an object. Indeed, such a guide allows the second measuring element to bear laterally against said protrusions, which makes it possible to ensure precision of the measurements done while avoiding lateral movements of the part of the second measuring element superimposed on another part of said second measuring element during the measurement of the circumference of an object. On the other hand, said second pair of protrusions forms a plane of reference corresponding to the zero graduation, which makes it possible, when taking a measurement, to read the value of the circumference of the measured object easily and precisely.

In one embodiment according to the invention, said first pair of protrusions can have curved free ends oriented toward said second graduated measuring element. This makes it possible to ensure that the part of said second measuring element is kept superimposed on another part of said second measuring element when measuring the circumference of an object.

Other embodiments of the device for measuring the circumference of bodily segments according to the invention are indicated in the appended claims.

The invention also relates to a kit for measuring the circumference of an object, in particular a limb, comprising:
at least one first graduated measuring element having a first longitudinal direction and defining a slide, and being arranged to be affixed along a bodily segment,
a plurality of second graduated measuring elements,
a plurality of sliders having a passage orifice defined by four walls, at least two of which are parallel to one another and which each form a guide wall of the slider along the slide so as to fit at least one second longitudinal graduated measuring element in a plane perpendicular to said first longitudinal direction, and
a plurality of straps.

Such a kit for measuring limb circumference comprises a small number of elements that can be assembled quickly and easily in order to have a device for measuring limb circumference as described above. This kit containing all of the essential elements of the present invention is small and can easily fit in a box having a format smaller than or equal to the format of a pocket of work clothing (for example, a laboratory apron).

Other embodiments of the kit for measuring the circumference of bodily segments according to the invention are indicated in the appended claims.

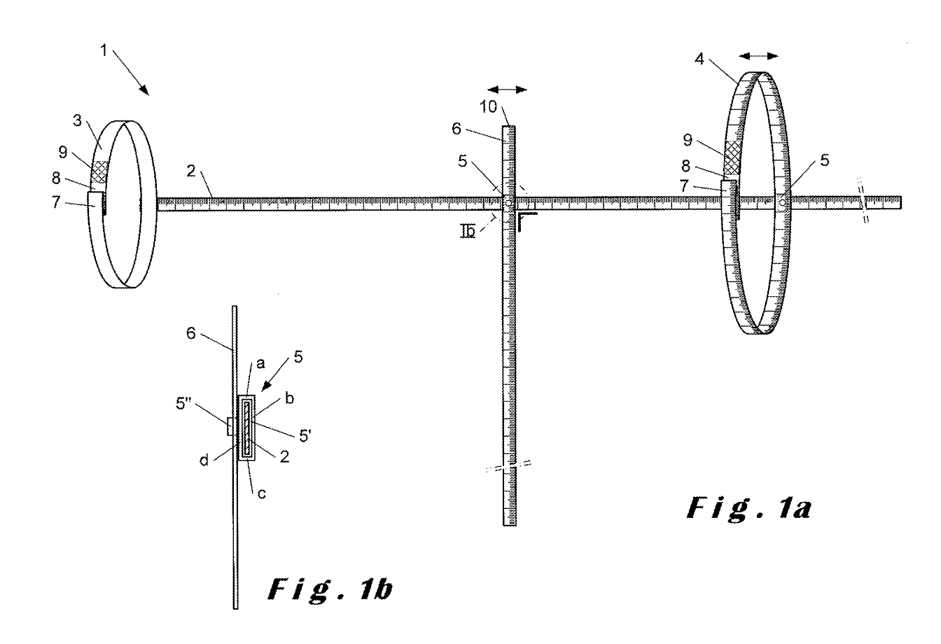

Other features, details and advantages of the invention will emerge from the description provided below, non-limitingly and in reference to the appended drawings. FIG. 1a is a diagrammatic view of a first embodiment of a device for measuring the circumference of bodily segments in its assembled form according to the invention. FIG. 1b is a detailed side view of a slider according to the invention, according to the zone indicated in FIG. 1a by the circle in broken lines.

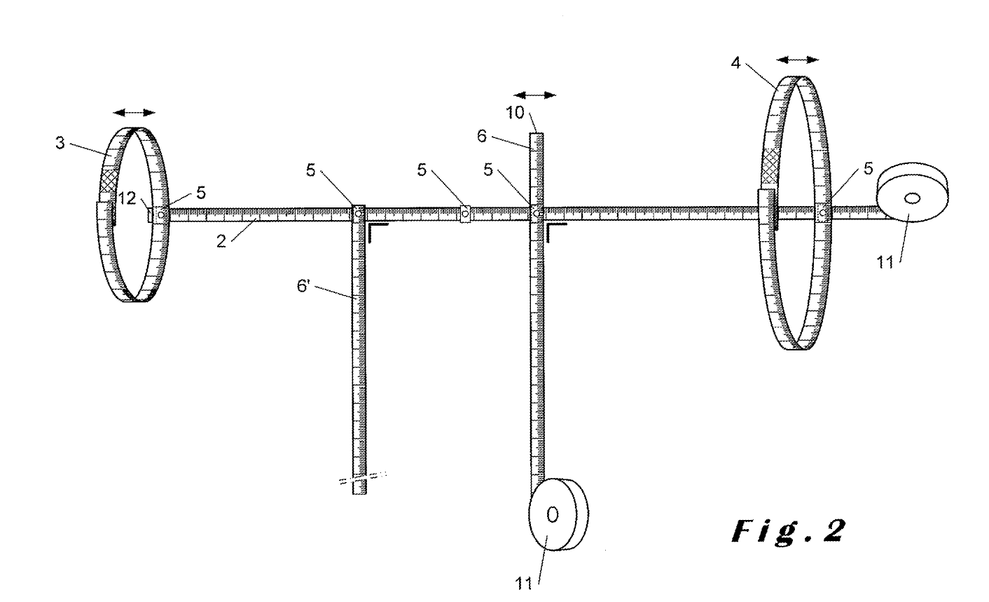

FIG. 2 is a diagrammatic view of another embodiment of a device for measuring the circumference of bodily segments in its full assembled form according to the invention.

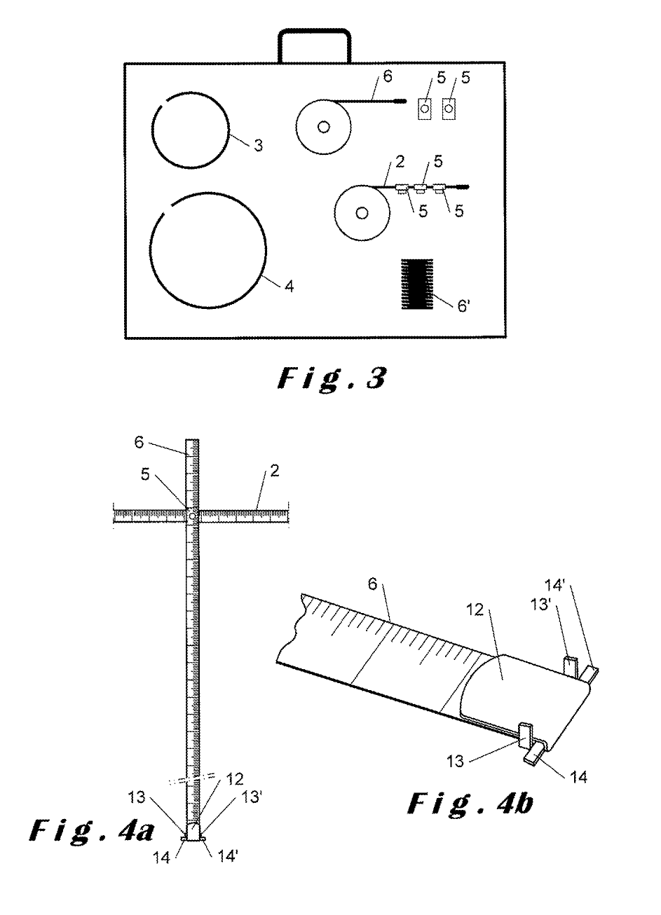

FIG. 3 is a diagrammatic view of an example of a kit for measuring the circumference of bodily segments according to the invention.

FIG. 4 illustrates an end-piece that can be present at one end of the graduated measuring element.

In the figures, identical or similar elements bear the same references.

FIG. 1a illustrates a first embodiment of a measuring device 1 for measuring the circumference of limbs in its assembled form. This measuring device 1 comprises a first longitudinal graduated measuring element or slide 2. It may for example be a tape measure made from flexible plastic or paper with a high tensile strength, like those used in the sewing field, or any other material having properties allowing it to follow the entire length of an object or a limb closely and with contact. This slide 2 forms a guide for sliders 5 that has a passage orifice (as shown in figure lb under number 5′) of said slide 2. A second longitudinal graduated measuring element 6 is connected to a slider 5, for example by fitting on a fastening means (as shown in figure lb under number 5″) present on said slider 5. The fastening means 5″ (see FIG. 1b) makes it possible to adjust said second longitudinal graduated measuring element 6 perpendicularly relative to said slide 2.

The measuring device 1 further comprises at least two straps 3, 4 positioned on either side of said slider 5, to which said second graduated measuring element 6 is connected, more particularly a distal strap 3 and a proximal strap 4 that are each connected to said slide 2. According to this first embodiment, the distal strap 3 is secured to said slide 2, on which it is for example glued or sewn, while the proximal strap 4 is connected to a slider 5 with a configuration identical to that to which said second graduated measuring element 6 is connected. The distal strap 3 is therefore stationary, while the proximal strap 4 and said second graduated measuring element 6 are movable and can be moved longitudinally along the slide 2 to the right or left (as indicated by the double arrows). Each of the straps 3, 4 further comprises a first end 7 and a second end 8, said first end 7 being folded down on said second end 8 in order to close said straps 3, 4 after positioning around a limb. As illustrated, several fastening zones 9 are provided on the straps 3, 4, such that the latter can be fastened around limbs having different circumferences.

Once both straps 3, 4 are correctly positioned such that said slide 2 is affixed to the surface of the limb in question, an operator can perform as many measurements as he wishes at any interval, for example every 2 cm, by simply moving the slider 5, to which said second graduated measuring element 6 is connected perpendicularly, along the slide 2. When the operator has moved said second graduated measuring element 6 to the location where he wishes to take a measurement of the circumference, this location corresponding to one of the graduations of the slide 2, he need only wind the second measuring element 6 around the limb to determine the circumference thereof corresponding to the measurement indicated by the graduation of the second graduated measuring element 6 located across from the zero graduation 10 of that same second graduated measuring element 6. According to this first embodiment, only the proximal strap 4 comprises graduations. Of course, the distal strap could also comprise graduations or, in one alternative embodiment according to the invention, each of the straps 3, 4 may have no graduations.

FIG. 1b illustrates, in detail and from the side, a slider 5 according to the invention. The slider 5 has a passage orifice 5′ delimited by four walls a, b, c, and d, which each form a guide wall for the slider 5 along the slide 2 so as to fit the second longitudinal graduated measuring element 6 into a plane perpendicular to the longitudinal direction of the slide 2.

FIG. 2 illustrates another embodiment of a device for measuring the circumference of bodily segments 1 in its assembled form. This figure copies the same elements as those described in FIG. 1. However, according to this additional embodiment, the measuring device 1 according to the embodiment comprises three additional sliders 5, one of which is connected to the distal strap 3, one of which is connected to an additional second graduated measuring element 6′ and one of which is not connected to any element. As illustrated, it is therefore possible for the two straps 3, 4 both to be connected to separate sliders 5, allowing them to be moved to the right or left (as indicated by the double arrows) longitudinally and perpendicularly along the slide 2. FIG. 2 also illustrates that the second graduated measuring elements 6, 6′ can indifferently have their end comprising the zero graduation 10 either along the upper edge of the slide 2 (case of the second graduated measuring element 6′), or past the upper edge of the slide 2 (case of the second graduated measuring element 6). The slider 5 to which no element is connected could for example allow the connection of an additional strap thereto, or a second additional graduated measuring element. According to this second embodiment, the second graduated measuring element 6 and the slide 2 are both measuring elements that can be wound and retracted using an automatic or manual winding system 11 for example comprising a return spring and a brake that can be actuated manually. Of course, the second graduated measuring element 6′ could also be connected to such a winding device 11.

This embodiment according to FIG. 2 is particularly advantageous, since all of the elements connected to a slider can be separated therefrom such that, after disassembly of the measuring device according to this example, the operator has two straps 3, 4, a slide 2 that can be wound in a winding device 11 and that comprises five sliders and two second graduated measuring elements, one 6 that can be wound in a winding device 11 and the other 6′ for example being able to be folded in the shape of an S. In order, following the winding of the slide 2, for the sliders 5 cooperating with the latter to remain positioned there, a removable or non-removable end-piece 12 can be provided at the end of said slide 2 to form a stop against which the sliders 5 are blocked such that they cannot “leave” said slide 2. The fact that this end-piece 12 is removable makes it possible, if needed, to add a slider 5 for an additional measuring element 6.

FIG. 3 illustrates a kit for measuring the circumference of bodily segments according to the invention, which [comprises] a first graduated measuring element 2, a plurality of sliders 5 (some positioned on the slide 2, others not), two second graduated measuring elements 6, 6′ (one folded in the shape of an S and the other wound in a winding device) and two straps 3, 4. Of course, the kit according to the invention is in no way limited to this example, and a kit that would comprise a given element in another number of copies is also an integral part of this invention.

FIGS. 4a and 4b illustrate a second graduated measuring element 6 provided at its end not connected to the first graduated measuring element 2 with a removable or non-removable end-piece 12 comprising a first pair of protrusions 13, 13′ extending upward in a direction perpendicular to the plane formed by said second graduated measuring element 6 and a second pair of protrusions 14, 14′ extending outwardly substantially in the same plane as that formed by said second graduated measuring element 6.

Of course, the present invention is in no way limited to the embodiments described above, and modifications may be made thereto without going beyond the scope of the appended claims.

1. A device for the iterative measurement of the circumference of an object, comprising:
a first longitudinal graduated measuring element having a first longitudinal direction, arranged to be affixed alongside said object and defining a slide,
a distal strap and a proximal strap fitted each in a plane substantially perpendicular to said longitudinal direction, and
a slider cooperating with said slide, connected to a second longitudinal graduated measuring element, said slider having a slide passage orifice and being positioned between the distal strap and the proximal strap,
wherein said slide is a flexible slide having a constant section over its entire length and in that said passage orifice is delimited by four walls, at least two of which are parallel to one another and which each form a guide wall for the slider along the slide so as to fit said second longitudinal graduated measuring element in a plane perpendicular to said first longitudinal direction.

2. The measuring device according to claim 1, wherein said slide and/or said second graduated measuring element can be wound.

3. The measuring device according to claim 1, wherein said slide and/or said second graduated measuring element is foldable, for example in an S shape.

4. The measuring device according to claim 1, wherein it comprises a plurality of sliders.

5. The measuring device according to claim 1, wherein said proximal strap is connected to the slide or to a slider other than that to which said second graduated measuring element is connected, said proximal strap being detachable or not.

6. The measuring device according to claim 1, wherein said passage orifice of said slider has a section similar to the section of the slide.

7. The measuring device according to claim 1, wherein said slide is a slide made from a flexible material, for example flexible plastic or paper with a sufficient tensile strength.

8. The measuring device according to claim 1, wherein said second measuring element connected to the slider is detachable.

9. The measuring device according to any one of claims. claim 1, wherein said distal strap is connected to the slide or to a slider other than that to which said second graduated measuring element is connected, said distal strap being detachable or not.

10. The measuring device according to any one of claims. claim 1, wherein said distal strap and/or said proximal strap is a graduated or not graduated longitudinal strip having first and second ends each provided with a closing means and having an open position and a closed position, said open position being a position where said first and second ends are spaced apart from one another, while the closed position is a position in which said first end of said longitudinal strip is folded down on said second end.

11. The measuring device according to claim 10, characterized in that wherein said closing means is chosen from the group made up of a press button, a pushbutton or self-gripping means, for example of the Velcro® type.

12. The measuring device according to claim 1, wherein said second measuring element is provided, at its end not connected to the first measuring element, with a removable or non-removable end-piece comprising a first pair of protrusions extending upward in a direction perpendicular to the plane formed by said second graduated measuring element and/or a second pair of protrusions extending outward substantially in the same plane as that formed by said second graduated measuring element.

13. A kit for measuring the circumference of an object, comprising:
at least one first graduated measuring element having a first longitudinal direction and defining a slide, and being arranged to be affixed along a bodily segment,
a plurality of second graduated measuring elements,
a plurality of sliders having a slider passage orifice defined by four walls, at least two of which are parallel to one another and which each form a guide wall of the slider along the slide so as to fit at least one second longitudinal graduated measuring element in a plane perpendicular to said first longitudinal direction, and
a plurality of straps.
